(19) United States
(12) Patent Application Publication
(10) Pub. No.: US 2009/0143618 A1
(43) Pub. Date: Jun. 4, 2009

(54) METHOD FOR THE PRODUCTION OF FORMIC ACID FORMATES

(75) Inventors: Alexander Hauk, Ludwigshafen (DE); Jorn Karl, Ludwigshafen (DE); Jurgen Paschold, Hochspeyer (DE); Stefan Gropp, Ludwigshafen (DE); Anna Valeska Lohmann, Limburgerhof (DE); Robert Lenz, Hochdorf-Assenheim (DE); Thomas Letzelter, Annweiler (DE); Robert Heinz, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF AKTIENGESELLSCHAFT, LUDWIGSHAFEN (DE)

(21) Appl. No.: 10/595,702

(22) PCT Filed: Nov. 5, 2004
(86) PCT No.: PCT/EP04/12543

§ 371 Date: May 5, 2006

(30) Foreign Application Priority Data
Nov. 6, 2003 (DE) 103 51 733.2
May 5, 2004 (DE) 10 2004 022 135.9

(51) Int. Cl. C07C 53/02

(52) U.S. Cl. 562/609

(57) ABSTRACT

A process is proposed for preparing acid formates in which
a liquid stream I comprising formic acid and
a liquid stream II comprising a metal formate
   are prepared,
the liquid streams I and II are fed to a rectification column in such a manner that a higher or identical feed point to the rectification column is chosen for the liquid stream II than for the liquid stream I,
the liquid streams I and II are mixed in the rectification column, with water being removed overhead from the rectification column and
a bottoms stream comprising the acid formate is taken off from the rectification column, which comprises the bottoms stream being produced as melt comprising less than 0.5% by weight of water.

The invention relates to a process for preparing acid formates and to the use of the acid formates prepared by the method for preserving and/or acidifying plant and/or animal materials, for treating biowastes and as an additive in animal nutrition or as a growth promoter for animals.

Acid formates have an antimicrobial action and are used, for example, for preserving and also for acidifying plant and animal materials, for instance grasses, agricultural products or meat, for treating biowastes or as an additive for animal nutrition.

Acid formates are compounds and mixtures which contain formate anions (HCOO⁻), cations (M^{x+}) and formic acid (HCOOH). They can be present together in the form of a solid or a liquid and may optionally comprise other components, for example other salts, additives or solvents, for instance water. Generally, the acid formates can be represented by the formula

HCOO⁻M^{x+}_{1/x}*y HCOH (I)

where M is a monovalent or polyvalent, inorganic or organic cation, x is a positive integer and denotes the charge of the cation and y gives the molar fraction of formic acid based on the formate anion. The molar fraction of formic acid based on the formate anion y is generally from 0.01 to 100, preferably from 0.05 to 20, particularly preferably from 0.5 to 5, and in particular from 0.9 to 3.1.

The nature of the inorganic or organic cation M^{x+} is in principle immaterial provided that said cation is stable under the conditions under which the acid formate is to be handled. This includes, for example, stability toward the reductive formate anion. Possible inorganic cations are the monovalent and/or polyvalent metal cations of the metals of groups 1 to 14 of the Periodic Table of the Elements, for example, lithium (Li⁺), sodium (Na⁺), potassium (K⁺), cesium (Cs⁺), magnesium (Mg²⁺), calcium (Ca²⁺), strontium (Sr²⁺) and barium (Ba²⁺), preferably sodium (Na⁺), potassium (K⁺), cesium (Cs⁺), and calcium (Ca²⁺). Possible organic cations are unsubstituted ammonium (NH₄⁺) and ammonium substituted by one or more carbon-containing radicals which may optionally also be bound to one another, for example methylammonium, dimethylammonium, trimethylammonium, ethylammonium, diethylammonium, triethylammonium, pyrrolidinium, N-methylpyrrolidinium, piperidinium, N-methylpiperidinium or pyridinium.

A carbon-containing organic radical is an unsubstituted or substituted aliphatic, aromatic or araliphatic radical having 1 to 30 carbon atoms. This radical can contain one or more heteroatoms, for instance oxygen, nitrogen, sulfur or phosphorus, for example —O—, —S—, —NR—, —CO—, —N═, —PR— and/or —PR₂ and/or be substituted by one or more functional groups which contain, for example, oxygen, nitrogen, sulfur and/or halogen, for example by fluorine, chlorine, bromine, iodide and/or a cyano group (the radical R in this case is likewise a carbon-containing organic radical). The carbon-containing organic radical can be a monovalent or polyvalent radical, for example divalent or trivalent radical.

A multiplicity of processes are known for preparing acid formates. In these processes, generally, a liquid stream I comprising formic acid and also a liquid stream II comprising a metal formate are prepared and said liquid streams I and II are mixed to obtain an acid-formate-containing product stream which is optionally further processed.

Such a process is disclosed, for example, by DE-A 102 37 379. According to this, the streams comprising formic acid and metal formate are combined, preferably in a column, which is advantageously operated in such a manner that a part of the solvent which is fed, generally water, is taken off. In this procedure, an acid-formate-containing bottom product having a water content of from 0.5 to 30% by weight, in particular having a water content of generally less than or equal to 1% by weight, can be obtained.

In contrast, despite the expected advantages in the further processing of the acid-formate-containing product stream, a person skilled in the art would hitherto not have known that it is possible to obtain the product stream having a water content which is markedly lower compared with known processes directly from the column in which the formic-acid-containing and metal-formate-containing streams are mixed.

Accordingly, a process has been found for preparing acid formates in which
a liquid stream I comprising formic acid and
a liquid stream II comprising a metal formate
   are prepared,
the liquid streams I and II are fed to a rectification column in such a manner that a higher or identical feed point to the rectification column is chosen for the liquid stream II than for the liquid stream I,
the liquid streams I and II are mixed in the rectification column, with water being removed overhead from the rectification column and
a bottoms stream comprising the acid formate is taken off from the rectification column, which comprises the bottoms stream being produced as melt comprising less than 0.5% by weight of water.

In a preferred embodiment of the process, a liquid stream I is prepared which comprises formic acid which is relatively highly concentrated, that is to say comprises at least 85% by weight of formic acid. Particularly preferably, the liquid stream I comprises at least 94% by weight, in particular 99% by weight, of formic acid. Advantageously in this case, formic acid types are used which are commercially available, for example formic acid having a purity of 85%, 94% or 99%.

The abovementioned liquid stream I comprising formic acid is preferably aqueous streams.

The inventors recognized that, when concentrated formic acid solutions are used, a lower residual water content can be obtained in the target product, the acid diformate, than is taken off as melt from the rectification column, in which the liquid streams I and II are mixed, in particular a residual water content of less than 0.3% by weight, preferably in the range of from 0.2 to 0.1% by weight, and in particular from 0.1 to 0.05% by weight.

The inventors have also recognized that, with the trend in decrease in water content in the liquid stream I, with otherwise unchanged conditions in the rectification column, the water content in the melt taken off via the bottom decreases.

The lower residual water content in the melt decisively influences the storage stability of the formulated end product acid diformate: the lower the residual water content in the end product, the lower the tendency to caking or lump formation.

A further advantage compared with a procedure having a higher water content in liquid stream I is that, to obtain a melt having the same, low water content, a lower number of theoretical plates, which is reduced in particular by about from 4 to 8 theoretical plates, is sufficient. Correspondingly, the rectification column can also be constructed with smaller size and thus lower capital and operating costs.

Preferably, the liquid streams I and II are each aqueous streams.

The process is not restricted with respect to the specific ways for preparing the liquid streams I and II. Preferably, these can be provided as described in DE-A 103 21 733 which is hereby explicitly incorporated in full by reference into the contents of the present application.

The liquid stream I can be obtained, for example, by partial hydrolysis of methyl formate, hereinafter denoted by the abbreviation MeFo (process stage (a) of DE-A 103 21 733) and removal by distillation of unreacted MeFo and methanol.

The liquid stream II can be obtained, for example, by the process variants described in DE-A 103 21 733:

According to this, an MeFo- and methanol-comprising stream can be converted, in a process stage c) into the metal-formate- and water-containing stream II by
i) reaction with a basic compound having a pKₐ of the conjugate acid of the corresponding dissociation state of ≧3, measured at 25° in aqueous solution, in the presence of water and
ii) removal of the methanol by distillation.

For a more detailed description of the process stage c), reference is made to said DE-A 103 21 733.

In a further alternative it is possible to produce the metal-formate-containing stream II by carbonylation of the corresponding metal hydroxide. Such a process is described in DE-A 102 37 380, the contents of which are hereby explicitly incorporated in full by reference into the present application.

In the next process step for preparing the acid formates, the prepared streams I, comprising formic acid, and II, comprising a metal formate, are mixed in a rectification column.

It is possible in this case, and possibly advantageous, to subject the formic-acid-containing liquid stream I and/or the metal-formate-containing liquid stream II, before the mixing in the rectification column, to a concentration in formic acid or in metal formate, in particular by removing a part of the water present by evaporation, preferably by distillation.

It has been found that it is essential, for the operation of the rectification column, to choose a higher or at least identical feed point for the liquid stream II than for the liquid stream I. The inventors have found that the presence of metal formate in the widest possible regions of the column, in particular in regions above the feed of the formic-acid-containing stream, is important for substantial removal of the water from the bottoms stream. It is essential in this case that the metal formate, in the system in the column, acts as entrainer for the formic acid.

Preference is given to an operation of the rectification column in which the bottom temperature in the rectification column is limited to a value below 135° C., in particular to a value below 125° C. For this, a person skilled in the art will set the overhead pressure in the column accordingly, taking into account familiar considerations, in particular the pressure drop occurring in the column.

In a preferred process variant, the feed point for the liquid stream II is chosen on or above the uppermost separation stage of the rectification column, with correspondingly lower capital costs.

In addition, or alternatively, the operating conditions in the column can be affected by the choice of the ratios of the liquid streams II and I: the ratio of the liquid streams II and I can be chosen in such a manner that the molar ratio of metal formate from the liquid stream II and formic acid from the liquid stream I is greater than 1, less than 1 or preferably between 0.95 and 1.05, particularly preferably equal to 1. As a result, the formic acid losses in the overhead stream of the rectification column can be limited, without an enriching part being required in the rectification column for this. In this case it is possible to take off virtually pure water.

In the selection of separating internals for the rectification column, it is advantageous to take into account internals of low pressure drop with simultaneously good separation efficiency, preferably ordered packings.

The number of theoretical plates of the rectification column is calculated according to general methods which are customary in the field. For the present separation task, generally preference should be given to from 5 to 15 theoretical plates.

The invention also relates to the use of the acid formates prepared by the inventive process for preserving and/or acidifying plant and/or animal materials, for treating biowastes, or as additives in animal nutrition and/or as growth promoter for animals.

Taking off the material of value, the acid formate, as a melt having a water content of less than 0.5% by weight, in the inventive process as soon as in the column in which the liquid streams comprising formic acid and metal formate are mixed gives significant economic advantages. In particular, for further workup of the melt taken off from the rectification column, apparatuses different from the known apparatuses for further processing product streams having a higher water content are required which are less complicated and less susceptible to faults, in particular cooling rollers or cooling belts, cooling plates or prilling towers. This decreases the capital costs compared with plants for conventional processes having a higher water content in the product stream.

Further processing steps, such as solid/liquid separations, crystallization and subsequent drying, which were required in known processes, are unnecessary.

The invention will be described in more detail hereinafter with reference to a drawing and exemplary embodiments.

In the drawings:

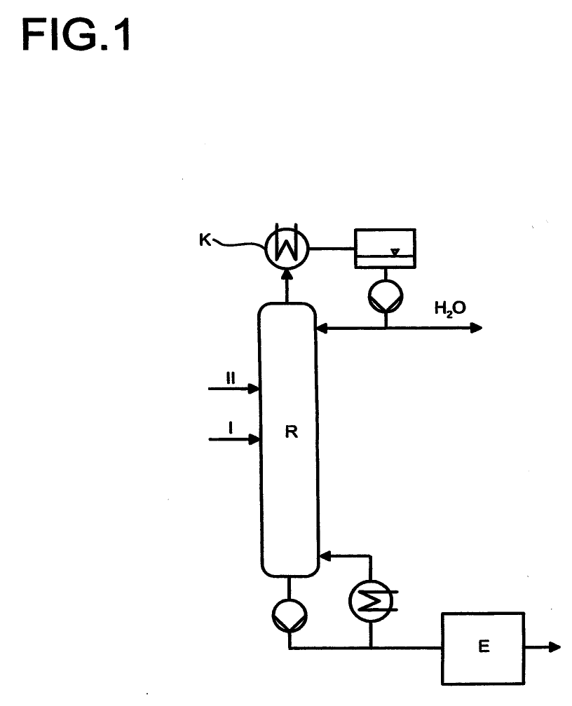

FIG. 1 shows the diagrammatic representation of a first embodiment of a plant for carrying out the inventive process and

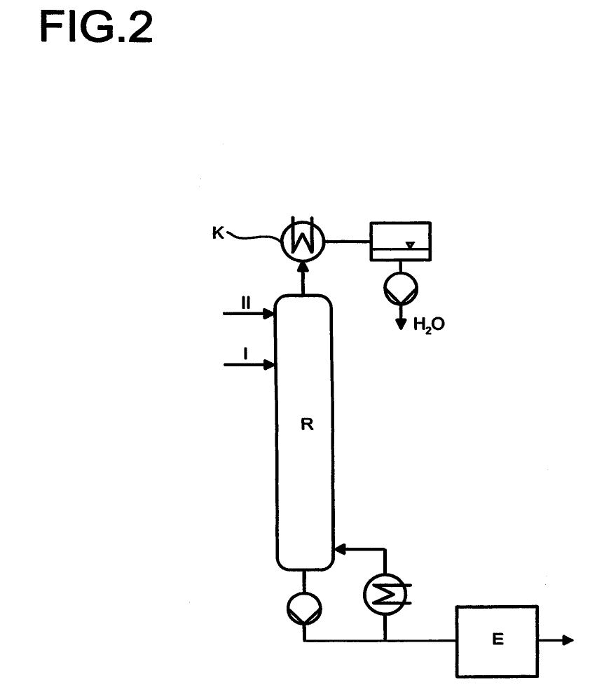

FIG. 2 shows the diagrammatic representation of a further preferred embodiment of a plant for carrying out the inventive process.

In the first embodiment of the inventive process shown diagrammatically in FIG. 1 a rectification column R is fed an aqueous stream II comprising a metal formate, and below same an aqueous stream I comprising formic acid. Above the feed of the aqueous stream II is disposed an enrichment part. In the rectification column R, the aqueous streams I and II are mixed and an overhead stream predominantly comprising water is taken off, which stream condenses in a condenser K at the column top, is in part reapplied to the column as reflux and the remainder is discharged. From the column bottom a melt comprising less than 0.5% by weight of water is taken off and is solidified in or on a downstream apparatus E. From the downstream apparatus E, optionally after treatment in a compacter which is not shown, the product of desired particle size is taken off.

The preferred embodiment shown in FIG. 2 differs from the embodiment in FIG. 1 by the aqueous stream II being applied to the uppermost tray of the rectification column R. In this embodiment, the rectification column R thus does not have an enrichment part. In this process variant, also, substantially pure water can be taken off at the top of the rectification column, provided that the ratio of the liquid streams II and I is chosen so that the molar ratio of metal formate from the liquid stream II and formic acid from the liquid stream I is greater than or equal to 1.

### Exemplary Embodiments

In a laboratory column which consisted of three or two sections each of diameter 30 mm each with 10 bubble cap trays, potassium diformate melts were produced.

The column was operated as a pure stripping column.

The feeds consisted of 75% strength aqueous potassium formate on the uppermost tray of the column and aqueous formic acid, five trays lower. Formic acid and potassium formate were supplied stoichiometrically.

Potassium diformate having differing water contents was discharged at the bottom, and at the top of the column, virtually pure water containing residues of formic acid of less than 1000 ppm was discharged.

In the examples, the formic acid concentration was varied in the feed. Furthermore, at each formic acid concentration, a first experiment (hereinafter denoted by the suffix A) was carried out at a higher overhead pressure, and a further experiment (hereinafter denoted by the suffix B) was carried out at a lower overhead pressure.

### EXAMPLE 1 A

The formic acid concentration in the feed was 30% by weight. The column consisted of three sections; the pressure drop over the column was approximately 35 mbar. The column was operated at an overhead pressure of 50 mbar. The bottom temperatures were between 132 and 135° C. In the melt in the column bottom, water contents of approximately 0.45% by weight were achieved.

### EXAMPLE 1 B

The formic acid concentration in the feed, the number of column sections and the pressure drop over the column were unchanged from example 1 A. The column was operated at an overhead pressure of 20 mbar and a bottom temperature between 122 and 127° C. A melt having a water content of approximately 0.35% by weight was taken off from the column bottom.

### EXAMPLE 2 A

The formic acid concentration in the feed was 85% by weight. The column consisted of two sections; the pressure drop over the column was approximately 25 mbar. The column was operated at an overhead pressure of approximately 35 mbar. At a bottom temperature of about 126° C., water contents between 0.18 and 0.2% by weight were achieved in the potassium diformate melt taken off from the column bottom.

### EXAMPLE 2 B

The formic acid concentration in the feed, the number of column sections and also the pressure drop over the column were unchanged from example 2 A. The overhead pressure was decreased to approximately 25 mbar. At a bottom temperature in the range from 124 to 126° C., water contents of from about 0.08 to 0.12% by weight were achieved in the potassium diformate melt taken off from the column bottom.

### EXAMPLE 3 A

The formic acid concentration in the feed was 94% by weight. The number of column sections and the pressure drop corresponded to example 2 A. The column was operated at an overhead pressure of approximately 35 mbar. At a bottom temperature of from 126 to 128° C., water contents of from about 0.08 to 0.1% by weight were achieved in the potassium diformate melt taken off from the column bottom.

### EXAMPLE 3 B

The formic acid concentration was unchanged from example 2 A. The number of column sections and also the pressure drop corresponded to example 2 A. The overhead pressure was decreased to approximately 25 mbar. At a bottom temperature of from 124 to 126° C., a water content of from about 0.05 to 0.07% by weight was achieved in the potassium diformate melt taken off from the column bottom.

### EXAMPLE 4 A

The formic acid concentration in the feed was 99% by weight. The number of column sections and also the pressure drop corresponded to example 2 A. The column was operated at an overhead pressure of approximately 35 mbar. At bottom temperatures of from 124 to 126° C., water contents of from about 0.05 to 0.08% by weight were achieved in the potassium diformate melt taken off from the column bottom.

### EXAMPLE 4 B

At an unchanged formic acid concentration in the feed, the overhead pressure was decreased to approximately 25 mbar. The number of column sections and also the pressure drop corresponded to example 2 A. The bottom temperatures and also the water content of the potassium diformate melt taken off from the column bottom were in the same range as described under example 3 A. At the high formic acid concentration used in the examples 3 A and 3 B, no significant effect of the overhead pressure was thus found on the water content in the melt taken off from the column bottom for a variation of the overhead pressure from 35 mbar to 25 mbar.

Examples 2 A, 2 B, 3 A, 3 B, 4 A and 4 B show that in the preferred process procedure using highly concentrated formic acid as starting stream I, particularly low water contents are achieved in the melt taken off from the column bottom.

1. A process for preparing acid formates in which
a liquid stream I comprising formic acid and
a liquid stream TI comprising a metal formate are prepared,
the liquid streams I and II are fed to a rectification column in such a manner that a higher or identical feed point to the rectification column is chosen for the liquid stream II than for the liquid stream I,
the liquid streams I and II are mixed in the rectification column, with water being removed overhead from the rectification column and
a bottoms stream comprising the acid formate is taken off from the rectification column,
which comprises the bottoms stream being produced as melt comprising less than 0.5% by weight of water.

2. A process as claimed in claim 1, wherein the content of liquid stream I of formic acid is at least 85% by weight.

3. A process as claimed in claim 2, wherein the content of liquid stream I of formic acid is at least 94% by weight.

4. A process as claimed in claim 1, wherein the liquid streams I and II are aqueous streams.

5. A process as claimed in claim 1, wherein the bottoms stream comprises less than 0.3% by weight of water, preferably between 0.2 and 0.1% by weight of water, particularly preferably from 0.1 to 0.05% by weight of water.

6. A process as claimed in claim 1, wherein the bottom temperature in the rectification column is limited to a value below 135° C.

7. A process as claimed in claim 6, wherein the bottom temperature in the rectification column is limited to a value below 125° C.

8. A process as claimed in claim 1, wherein the feed point for the liquid stream TI is chosen on or above the uppermost separation stage of the rectification column.

9. A process as claimed in claim 1, wherein the ratio of the liquid streams II and I is chosen in such a manner that the molar ratio of metal formate from the liquid stream II and formic acid from the liquid stream I is in the range from 0.95 to 1.05, preferably 1.

10. A process as claimed in claim 1, wherein the rectification column is fitted with separating internals of low pressure drop, preferably with ordered packings.

11. A process as claimed in claim 1, wherein the number of theoretical plates of the rectification column is chosen from 5 to 15.

12. A process as claimed in claim 3, wherein the content of liquid stream I of formic acid is at least 99% by weight.

13. A process as claimed in claim 5, wherein the bottoms stream comprises between 0.2 and 0.1% by weight of water.

14. A process as claimed in claim 13, wherein the bottoms stream comprises from 0.1 to 0.05% by weight of water.

15. A process as claimed in claim 9, wherein the molar ratio of metal formate from the liquid stream TI and formic acid from the liquid stream I is 1.

16. A process as claimed in claim 10, wherein the rectification column is fitted with ordered packings.
